(19) United States
(12) Patent Application Publication
(10) Pub. No.: US 2009/0075867 A1
(43) Pub. Date: Mar. 19, 2009

(54) Triazine compounds and compositions thereof for the treatment of cancers

(76) Inventors: Lyne Gagnon, Laval (CA); Boulos Zacharie, Laval (CA); Christopher Penney, Pierrefonds (CA)

Correspondence Address: NIXON & VANDERHYE, PC, 901 NORTH GLEBE ROAD, 11TH FLOOR, ARLINGTON, VA, 22203, US

(21) Appl. No.: 11/919,984

(22) PCT Filed: May 19, 2006
(86) PCT No.: PCT/CA2006/000832

§ 371 Date: Nov. 6, 2007

Related U.S. Application Data
(60) Provisional application No. 60/682,374, filed on May 19, 2005.

(51) Int. Cl. A61K 31/53; A61P 35/00; A61K 31/704; A61K 31/675; A61K 38/00; A61K 31/7048; A61K 31/7068

(52) U.S. Cl. 514/8; 514/245; 514/34; 514/110; 514/27; 514/49

(57) ABSTRACT

Compounds useful in the treatment of metastatic melanoma and other cancers containing a triazine ring scaffold are described. These compounds may be classified into two groups: (1) two disubstituted triazine rings are covalently linked by an organic linker to each other and (2) one trisubstituted triazine ring.

### CROSS-REFERENCE TO RELATED APPLICATION

This application claims the benefit of provisional U.S. Appln. No. 60/682,374, filed May 19, 2005.

### FIELD OF THE INVENTION

The present invention relates to the treatment of metastatic melanoma and certain other cancers with novel organic compounds which contain a triazine ring scaffold. These compounds may be classified into two groups. In the first group of compounds, two disubstituted triazine rings are covalently linked by an organic linker to each other. The second group of compounds consists of one trisubstituted triazine ring.

### BACKGROUND OF THE INVENTION

Cancer refers to more than one hundred clinically distinct forms of the disease. Almost every tissue of the body can give rise to cancer and some can even yield several types of cancer. Cancer is characterized by an abnormal growth of cells which can invade the tissue of origin or spread to other sites. In fact, the seriousness of a particular cancer, or the degree of malignancy, is based upon the propensity of cancer cells for invasion and the ability to spread. That is, various human cancers (e.g., carcinomas) differ appreciably as to their ability to spread from a primary site or tumor and metastasize throughout the body. Indeed, it is the process of tumor metastasis which is detrimental to the survival of the cancer patient. A surgeon can remove a primary tumor, but a cancer that has metastasized often reaches too many places to permit a surgical cure. To successfully metastasize, cancer cells must detach from their original location, invade a blood or lymphatic vessel, travel in the circulation to a new site, and establish a tumor.

The twelve major cancers are prostate, breast, lung, colorectal, bladder, non-Hodgkin's lymphoma, uterine, melanoma, kidney, leukemia, ovarian, and pancreatic cancers. Melanoma is a major cancer and a growing worldwide health problem by virtue of its ability to metastasize to most organs in the body which includes lymph nodes, lungs, liver, brain, and bone. The clinical outcome for patients with metastasis to distant sites is significantly worse than that seen with regional lymph node metastases. The median survival time for patients with lung metastases is eleven months while that for patients with liver, brain, and bone metastases is four months. Four types of treatment have been used for distant melanoma metastases: surgery, radiation therapy, chemotherapy, and immunotherapy. Surgery is most often used to improve the quality of life of the patient, such as removing a metastasis that is obstructing the gastrointestinal tract. Radiation therapy has some degree of efficacy in local control of metastases, but is primarily limited to cutaneous and/or lymph node metastases. A number of chemotherapeutic agents have been evaluated for the treatment of metastatic melanoma. However, only two cytotoxic drugs are able to achieve a response rate of 10% or more. These drugs are decarbazine (DTIC) and nitrosoureas. Only DTIC is approved for the treatment of melanoma in most countries. Subsequently, the lack of clinically significant beneficial long term effects or surgery, radiation therapy, and chemotherapy for the treatment of metastatic melanoma has led to the use of immunotherapy. Thus far, most attention has been given to the cytokines interleukin-2 and interferon-α. Clinical trials have yielded better results with interleukin-2 but, on average, only 15% of patients with metastatic melanoma exhibit a significant reduction in tumor burden in response to interleukin-2. Recently, a phase III clinical trial was completed for the treatment of metastatic melanoma with interleukin-2 and histamine dihydrochloride, but statistical significance was not achieved as compared to interleukin-2 alone, and this treatment awaits U.S. FDA approval. A need therefore exists for compounds which are efficacious, preferably with reduced toxicity, for the treatment of melanoma.

Other cancers may be more effectively treated with chemotherapeutic agents than melanoma. Chemotherapeutic agents suffer, however, from two major limitations. First, the chemotherapeutic agents are not specific for cancer cells and particularly at high doses, they are toxic to normal, rapidly dividing cells. Second, sooner or later, cancer cells develop resistance to chemotherapeutic agents thereby providing no further benefit to the cancer patient. As described above for melanoma, other treatment modalities have been explored to address the limitations imposed by the use of chemotherapeutic agents. But, as noted above for the treatment of melanoma, surgery (e.g., the inability to completely remove extensive metastases), radiation (e.g., the inability to selectively deliver radiation to cancer cells), and immunotherapy (e.g., the use of toxic cytokines with limited efficacy) have been of limited success for the treatment of other cancers. For this reason, other newer therapeutic approaches are under exploration (e.g., antiangiogenesis agents, gene therapy) but these treatments are, relatively speaking, in their infancy. Therefore, as with melanoma, a need still exists for novel compounds, which are efficacious (e.g., reducing tumor size or spread of metastases) and have reduced toxicity, for the treatment of other cancers.

### SUMMARY OF THE INVENTION

It is an objective of the present invention to provide drugs with a novel mechanism of action or biochemical target, but reduced toxicity, for the treatment of at least some cancers, especially metastatic melanoma. With the judicious choice of an appropriate biochemical target important to the survival of the cancer cell and when used in combination with other standard but reasonably efficacious compounds, it then becomes possible to provide a novel, more durable (i.e., less susceptible to drug resistance), less toxic therapy for the treatment of cancer. Such a novel biochemical target is described in one embodiment of the present invention for it has been surprisingly discovered that compounds which can bind to human immunoglobulins, as a biochemical target, have significant anticancer activity. Furthermore, this binding to human antibody is not deleterious to normal cellular function and so cancer therapy with the compounds of the present invention is relatively nontoxic, especially in comparison with standard drugs routinely used for cancer therapy.

Further aspects of the invention will be apparent to a person skilled in the art from the following description and claims and generalizations thereto.

### BRIEF DESCRIPTION OF THE DRAWINGS

FIG. 1 shows the antitumor efficacy of compound 2 or doxorubicin (Dox) on B16F10 primary tumors.

FIG. 2 shows the antitumor efficacy of compounds 1, 2, 8, and doxorubicin (Dox) on B16F10 primary tumors.

FIG. 3 shows the antimetastatic efficacy of compounds 1, 2, 8 and 10 on metastasis of B16F10 lung tumors.

FIG. 4 shows the antimetastatic efficacy of compounds 1, 2, doxorubicin (Dox), and Dox plus compound 2 on B16F10 lung metastases.

FIG. 5 shows the antimetastatic efficacy of compound 14 and doxorubicin (Dox) on B16F10 lung metastases.

FIG. 6 shows the antitumor efficacy of compounds 1, 2, 3, 8, cyclophosphamide (CY), and CY plus compound 2 on DA-3 breast tumors.

FIG. 7 shows the antitumor efficacy of compound 14 and cyclophosphamide (CY) on DA-3 breast tumors.

FIG. 8 shows the antitumor efficacy of intratumoral injection of compound 2, cyclophosphamide (CY), and CY plus compound 2 on DA-3 breast tumors.

FIG. 9 shows the antitumor efficacy of intratumoral injection of compound 2, cyclophosphamide (CY), and CY plus compound 2 on DA-3 breast tumors. Tumor weights (FIG. 9A) and volumes (FIG. 9B) at the end of the trial are shown.

FIG. 10 shows the antitumor efficacy of compound 2, as compared to acetylsalicylic acid on P815 primary tumors.

FIG. 11 shows the antitumor efficacy of compounds 3, 14, and 19, as compared to acetylsalicylic acid on P815 primary tumors.

FIG. 12 shows the antitumor efficacy of oral administration of compound 2 and acetylsalicylic acid on P815 primary tumors.

FIG. 13 shows the antitumor efficacy of compounds 1 and 2, as compared to cyclophosphamide (CY) on xenograft human prostate PC-3 tumors.

FIG. 14 shows the antitumor efficacy of compound 8 and cyclophosphamide (CY) on xenograft human prostate PC-3 tumors.

FIG. 15 shows the antitumor efficacy of compounds 13 and 19, as compared to cyclophosphamide (CY) on xenograft human prostate PC-3 tumors.

### DETAILED DESCRIPTION OF CERTAIN EMBODIMENTS OF THE INVENTION

Compounds of the present invention, or pharmaceutically acceptable derivatives thereof, are described by the following two formulas which represent dimeric triazine compounds, or compounds with two triazine rings, and monomeric triazine compounds, or compounds with one triazine ring.

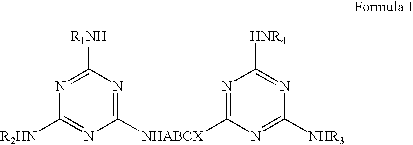

where A=—(CH₂)ₙ—, n=0, 1, 2, 3 or

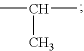

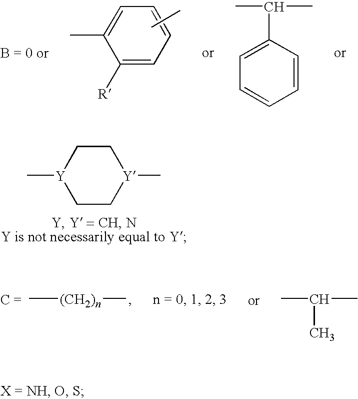

R′=hydrogen or C₁₋₄ alkyl, C₁₋₄ N-methylaminoalkyl or N,N-dimethylaminoalkyl;

A is not necessarily equal to C;

and wherein R₁, R₂, R₃ and R₄ are independently selected from the group consisting of hydrogen, C₂₋₆ alkyl or alkenyl, C₂₋₆ hydroxyalkyl, C₂₋₆ aminoalkyl, trifluoromethyl, pentafluoroethyl, phenyl, naphthyl, benzyl, biphenyl, phenethyl, piperazinyl, N-methylpiperazinyl, N-methylpiperazinyl, morpholinyl, piperidinyl, methylpiperidinyl, ethylpiperidinyl, indenyl, 2,3-dihydroindenyl, C₄-C₇ cycloalkyl or cycloalkenyl, indoyl, methylindoyl, ethylindoyl, and substituted five-membered aromatic heterocyclic rings of the following formulas:

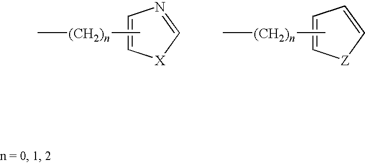

X is defined as above and Z=NH or CH₂
or substituted phenyl rings of the following formulas:

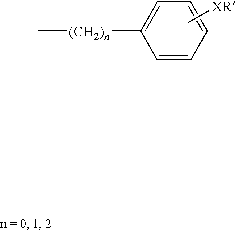

X and R′ are defined as above

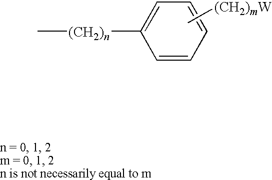

W=hydrogen, CH₃, NH₂, COOR′, or OR′

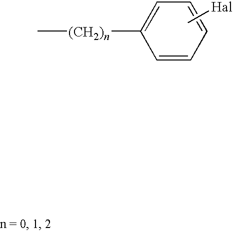

### Hal=Halogen (F, Cl, etc.)

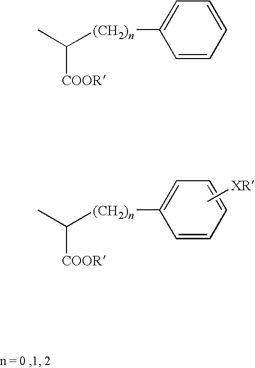

X and R′ are defined as above.

In one embodiment of the present invention, there are provided disubstituted triazine dimers in which each triazine monomer is connected to the other by an organic linker wherein said linker contains a 1,3- or 1,4-substituted phenyl group. That is,

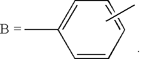

In such cases, it is possible for A=C=0 and the phenyl group becomes the linker which connects the two triazine monomers. In such a case, the general formula becomes:

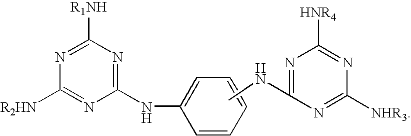

This represents one preferred embodiment of the present invention when A=C=0 but another preferred embodiment is provided when A=—(CH₂)ₙ—, where n=1 or 2 while C=0, or A=0 while C=—(CH₂)ₙ— where n=1 or 2, or A=C═—(CH₂)ₙ— where n=1 or 2. Thus, for example, a preferred embodiment of the present invention is A=—(CH₂)₂— and C=0, or A=0 and C═—(CH₂)₂—. In one preferred embodiment, the general formula becomes:

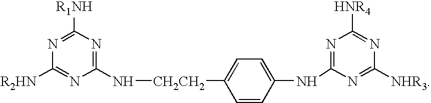

In an alternative embodiment of the present invention, no phenyl group is present in the organic linker which connects the two disubstituted triazine rings, or B=0. That is, the triazine dimers are connected by an alkyl chain. Thus, for example, another preferred embodiment of the present invention is A=C═—CH₂— and B=0. Therefore, the organic linker contains a —CH₂CH₂— or ethylene group and the general formula becomes:

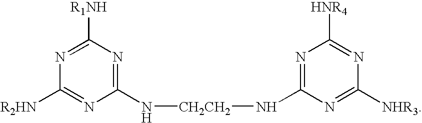

Regardless of the organic linker which connects the two triazine rings, it is a preferred embodiment of the present invention that R₁, R₂, R₃ and R₄ are defined as follows:

R₁=hydroxyethyl, hydroxypropyl, hydroxybutyl
=aminoethyl, aminopropyl, aminobutyl
=phenyl, anilino, hydroxyphenyl

R₂=phenethyl, hydroxyphenethyl, aminophenethyl
=hydroxyethyl, hydroxypropyl, hydroxybutyl

R₃=phenyl, anilino, hydroxyphenyl

R₄=fluorophenyl, phenyl, anilino, hydroxyphenyl
=hydroxyethyl, hydroxypropyl, hydroxybutyl.

In one embodiment of the present invention, R₁, R₂, R₃ and R₄ are not all the same (i.e., at least one, two, or three are different from the others). In another embodiment of the present invention, at least one, two, three, or all four of R₁, R₂, R₃ and R₄ is a phenyl ring or a substituted phenyl ring.

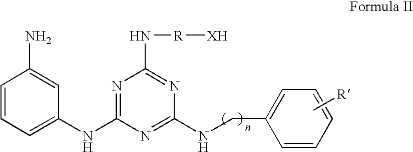

where R═—(CH₂)ₚ—, p=2-6 or

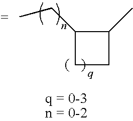

### X═NH, O, or S

### and R′=NH₂, OCH₃, F or Cl.

In another embodiment of the present invention, the group —R—XH may be replaced by

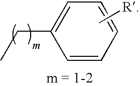

In such a case, the general formula becomes:

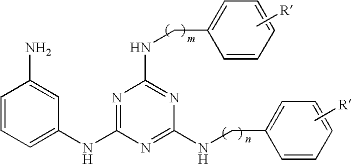

wherein R′ is defined as above but, if two R′ substituents are present in the same compound, both R′ substituents may be the same (amino, methoxy, fluorine) or one R′ substituent can be an amino group or fluorine atom while the second is a methoxy group. Also, m and n are defined as above but it is not necessary that m is equal to n.

In still another embodiment of the present invention, in the case where R′ is meta amino and n=0 then —R—XH may be replaced such that the general formula is:

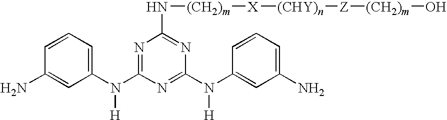

wherein m=1-2, n=2-4, X═CHY, O, or S; Y═H or OH; and Z=zero, O, or S.

Finally, in still another embodiment of the present invention, the group —R—XH may be replaced by a hydrogen atom. In such a case, the general formula becomes:

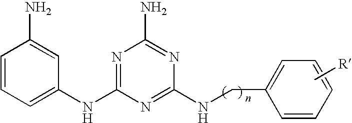

wherein R′ and n are defined as above.

When m is equal to n and R′ is an amino or methoxy group or fluorine atom, then the compound becomes a bis(alkaryl) substituted triazine (m=n=1 or 2). This symmetric substitution does not, however, represent a preferred embodiment of the present invention. A preferred embodiment of the present invention is provided by the bis(aryl) substituted triazine that results when n=0 and the corresponding R′=meta NH₂. The latter is less susceptible to oxidation.

Regardless of the structure defined above, it is a preferred embodiment of the present invention that R′ is an amino group. More preferred is that the amino group is located at the meta position. Less preferred is that the amino group is located at the ortho position because of its reduced bioactivity and increased susceptibility to oxidation.

Particularly preferred are the following compounds:

| Compound No. | Structure |
|---|---|
| 1 | 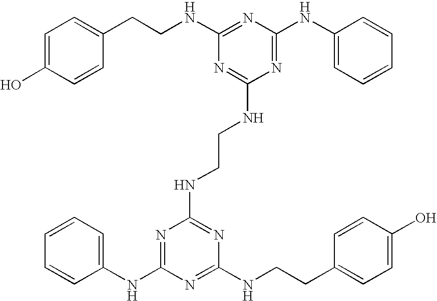 |
| 2 | 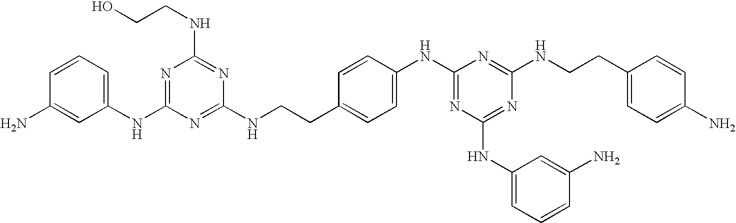 |
| 3 | 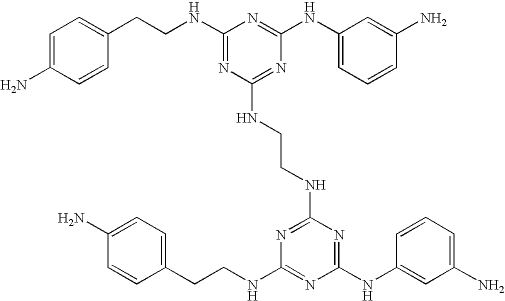 |
| 4 | 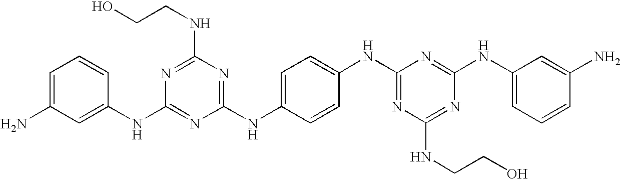 |
| 5 | 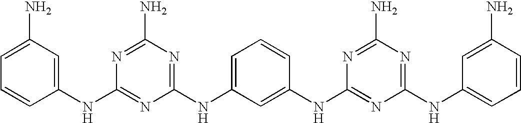 |
| 6 | 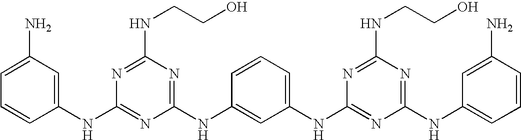 |
| 7 | 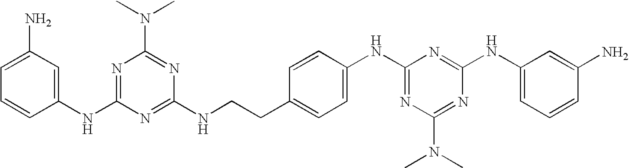 |
| 8 | 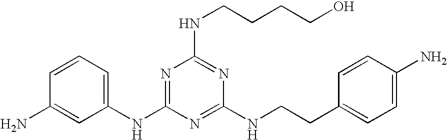 |
| 9 | 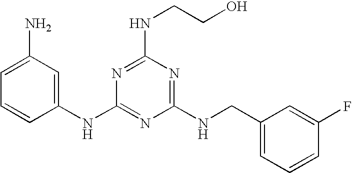 |
| 10 | 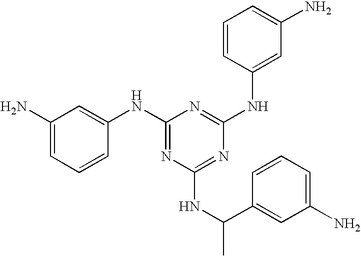 |
| 11 | 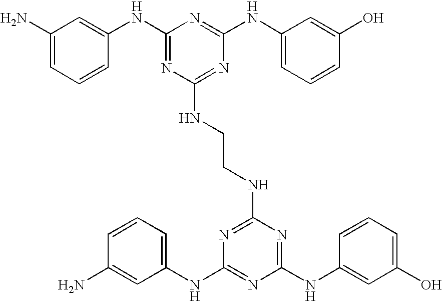 |
| 12 | 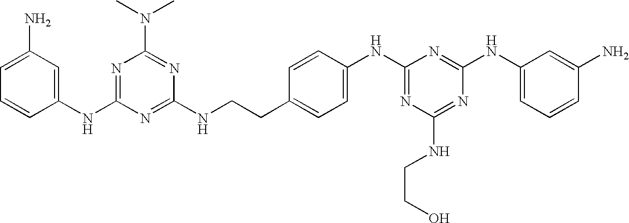 |
| 13 | 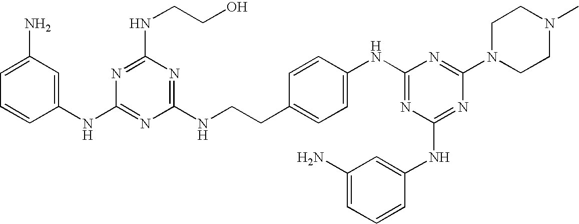 |
| 14 | 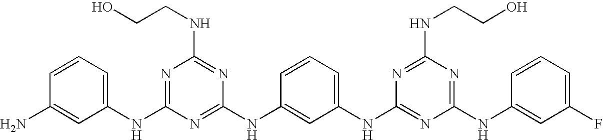 |
| 15 | 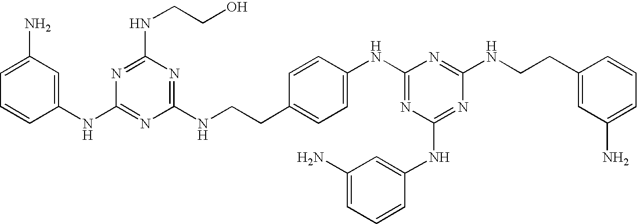 |
| 16 | 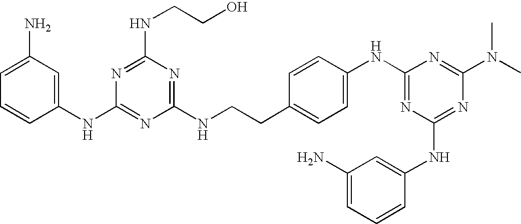 |
| 17 | 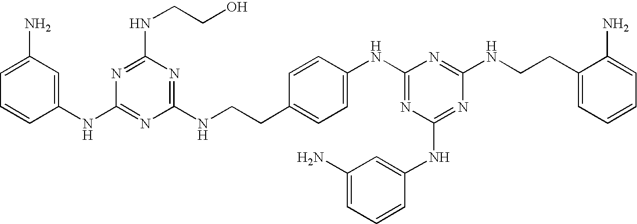 |
| 18 | 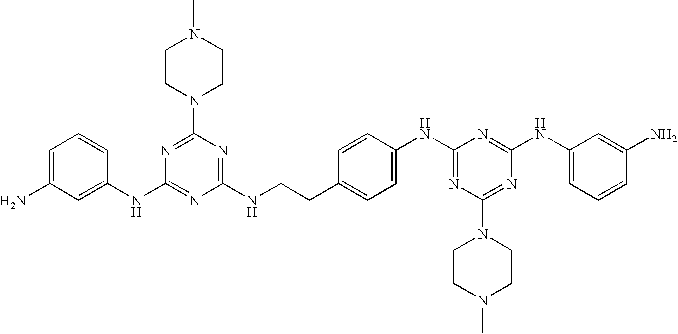 |
| and 19 | 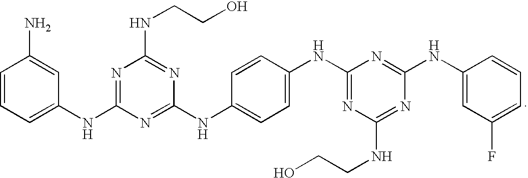 |

Specific compounds of the above formulas, their synthesis, and characterization are described in Int'l Patent Application No. PCT/CA2004/002003 “Dimer Triazine Compounds for the Treatment of Autoimmune Diseases” (compounds 5 and 7 are novel species of the generic compound disclosed therein) and Int'l Patent Application No. PCT/CA2005/001344 “Compounds which Bind to the Tail (Fc) Portion of Immunoglobulins and their Use.” Said compounds described in these two applications have also been shown to bind to human immunoglobulins, especially the tail or Fc portion of IgG. This was demonstrated by a competitive protein A binding ELISA in which compounds of the present invention would compete with human IgG for binding to bacterial protein A and by the ability of these compounds, when covalently linked to a solid-phase matrix, to bind and extract from solution human and mouse IgG. Therefore, the common apparent biochemical target which results in the anticancer activity of these compounds is IgG or, more broadly, immunoglobulin-like protein. Some support for the notion that IgG antibody could represent a biochemical target for subsequent anticancer activity is provided in U.S. Pat. No. 5,189,014 in which it was demonstrated that administration of bacterial protein A to rats with lung metastases resulted in a significant reduction in the number of metastases. Bacterial protein A can bind to the tail portion of most antibodies. For example, protein A will bind to IgG1, IgG2, and IgG4 immunoglobulins. Protein A is not cytotoxic to cancer cells but is toxic to humans; therefore, it cannot be used in vivo as a drug for the treatment of human cancers. It has not been previously discovered that low molecular weight compounds (<1,000; for comparison, protein A is 42,000), which bind to antibodies and are cytotoxic to cancer cells, can be administered in vivo for the treatment of cancer. In fact, the majority of compounds used, or in development, for the treatment of cancer bind to an enzyme, receptor, or DNA. The problem is that while these biochemical targets may be more active or over-expressed in cancer cells as compared to normal cells, they are not restricted to cancer cells. Subsequently, the majority of compounds, especially chemotherapeutic agents, are toxic since they interfere with biochemical targets that are important for normal cell proliferation and function. Again, this is particularly problematic with highly proliferating normal cell populations.

Although the compounds of the present invention bind to antibodies and are cytotoxic to cancer cells, it does not preclude the possibility that the anticancer activity arises from the effect that results when the antibodies, with bound compound, bind by their tail (Fc) portion to their respective Fc receptors. Fc receptors are glycoproteins which can be found in the systemic circulation (soluble receptors) or which can be present on the surface of normal or some cancer cells. Fc receptors include FcγRI (CD64), FcγRII (CD32), and FcγRIII (CD16) which will bind to IgG. Thus, for example, Cassard et al. Immunology Letters 75:1-8 (2000) reported that Fc receptors that bind to IgG are present on a human metastatic melanoma cell and suggested that these receptors play a role in the migration of tumor cells and/or metastasis formation. Indeed, Eshel et al. Cancer Biology 12:139-147 (2002) stated that Fc receptor expression on tumor cells is associated with a more tumorigenic phenotype which binds host antitumor antibodies. A simple hypothesis is that Fc receptors on tumor cells sequester host antibodies and dampen the immune response. If correct, this hypothesis suggests that compounds could protect antitumor antibodies by interfering with their ability to bind tightly enough to tumor Fc receptors. Similarly, the compounds could also protect antitumor antibodies from being tightly bound by nontumor (soluble or normal cell) Fc receptors. On the other hand, the anticancer effect of the compounds of the present invention may be more subtle in that they do not significantly alter the binding affinity of the Fc portion of the antibody with its receptor but instead alter or dampen signal transduction that may occur, and subsequent cellular activation, upon binding of the antibody to the tumor Fc receptor. Some support for the above is provided by Gillies et al. Cancer Research 59:2159-2166 (1999) and their observation that the efficacy of an antibody-interleukin 2 fusion protein was improved by reducing the binding to Fc receptors.

The above suggests that there may be a correlation between Fc receptor expression on the tumor cell and the ability of the compounds of the present invention to exert an antitumor effect. As noted above, Fc receptors are present on the surface of metastatic melanoma cells. If, as also noted above, these cancer cell-surface Fc receptors function not only to sequester host antibodies but are necessary for cancer cells to proliferate and invade (e.g., more tumorigenic phenotype), then the cytotoxicity of the compounds of the present invention becomes understandable. This also helps to explain the selective cytotoxicity of these compounds towards cancer cells but not normal cells. In the latter case, cell-surface Fc receptors serve primarily to bind antibodies and are not extensively involved in cell proliferation. The corollary which follows is that the compounds of the present invention would not be effective against all cancers nor able to kill every last cancer cell within a tumor. Lethality would be dependent upon the maturity of each cancer cell within the tumor or, collectively, the evolution of the phenotype (e.g., up-regulation of Fc receptor expression) of the tumor. Indeed, this expectation is borne out in the examples provided herein whereby treatment of an animal with a primary or metastatic tumor fails to completely eliminate the cancer. While it is within the scope of the invention to use the compounds as a monotherapy for the treatment of cancer, it is a preferred embodiment of the present invention that the compounds be used in combination with already approved but more toxic anticancer agents (e.g., chemotherapeutic agents, cytokines, radiation therapy, etc.). Examples of chemotherapeutic agents which may be used with the compounds of the present invention include decarbazine, doxorubicin, daunorubicin, cyclophosphamide, busulfex, busulfan, vinblastine, vincristine, bleomycin, etoposide, topotecan, irinotecan, taxotere, taxol, 5-fluorouracil, methotrexate, gemcitabine, cisplatin, carboplatin, and chlorambucil. Examples of cytokines which may be used with the compounds of the present invention include interleukin-2 and interferon (e.g., alpha, beta, and gamma). Thus, for example, in a particularly preferred embodiment of the present invention, the compounds may be used with interleukin-2 (with or without histamine and/or low dose cyclophosphamide) or with decarbazine (DTIC) for the treatment of metastatic melanoma. In another embodiment of the invention, the compounds may be used to change the availability of treatment with chemotherapeutic agents by increasing efficacy of such an agent at lower, less toxic doses.

Compounds of the present invention include all pharmaceutically acceptable derivatives, such as salts and prodrug forms thereof, and analogues as well as any geometrical isomers or enantiomers. Formulations of the active compound may be prepared so as to provide a pharmaceutical composition in a form suitable for enteral, mucosal (including sublingual, pulmonary, and rectal), parenteral (including intramuscular, intradermal, subcutaneous, and intravenous), or topical (including ointments, creams, or lotions) administration. In particular, compounds of the present invention may be solubilized in an alcohol or polyol solvent (e.g., solutol HS 15 (polyethylene glycol 660 hydroxystearate from BASF), glycerol, ethanol, 5% dextrose, etc.) or any other biocompatibile solvent such as cremophor EL (also from BASF), dimethyl sulfoxide (DMSO), or dimethylacetamide. The formulation may, where appropriate, be conveniently presented in discrete dosage units and may be prepared by any of the methods well-known in the art of pharmaceutical formulation. All methods include the step of bringing together the active pharmaceutical ingredient with liquid carriers or finely divided solid carriers or both as the need dictates. When appropriate, the above-described formulations may be adapted so as to provide sustained release of the active pharmaceutical ingredient. Sustained release formulations well-known to the art include the use of a bolus injection, continuous infusion, biocompatible polymers, or liposomes.

Suitable choices in amounts and timing of doses, formulation, and routes of administration can be made with the goals of achieving a favorable response in the mammal (i.e., efficacy), and avoiding undue toxicity or other harm thereto (i.e., safety). Therefore, “effective” refers to such choices that involve routine manipulation of conditions to achieve a desired effect: e.g., total or partial response as evidenced by factors which include reduction in tumor burden and/or tumor size as well as increase in survival time and/or quality of life which is associated with a reduction in amount and/or duration of treatment with standard but more toxic anticancer agents.

The amount of compound administered is dependent upon factors such as, for example, bioactivity and bioavailability of the compound (e.g., half-life in the body, stability, and metabolism); chemical properties of the compound (e.g., molecular weight, hydrophobicity, and solubility); route and scheduling of administration; and the like. It will also be understood that the specific dose level to be achieved for any particular patient may depend on a variety of factors, including age, health, medical history, weight, combination with one or more other drugs, and severity of disease.

The term “treatment” or “treating” refers to, inter alia, reducing or alleviating one or more symptoms of autoimmune disease in a mammal (e.g., human) affected by disease or at risk for developing disease. For a given patient, improvement in a symptom, its worsening, regression, or progression may be determined by an objective or subjective measure.

Finally, it will be appreciated by those skilled in the art that the reference herein to treatment extends to prophylaxis as well as therapy of an established cancer. Thus, for example, compounds of the present invention could be used after surgical removal of the primary tumor or prior to surgery or aggressive chemotherapy or even when the patient is in remission. The relative lack of toxicity of the compounds when compared to standard cancer therapies allows for a more liberal prophylactic use than would be advisable with standard therapies. The dose to be administered will ultimately be at the discretion of the oncologist. In general, however, the dose will be in the range from about 1 to about 100 mg/kg per day. More preferably, the range will be between 2 to 50 mg/kg per day. The dosage unit per day may be 10 mg or more, 100 mg or more, 10 g or less, 20 g or less, or any range therebetween.

### EXAMPLES

The following examples further illustrate the practice of this invention but are not intended to be limiting thereof.

### Example 1

### In Vitro Cytotoxicity of Compounds Assayed on Normal and Cancer Cells

This assay was performed to determine the effect of compounds of the present invention on cell cytotoxicity. Cells were incubated in presence or absence of compounds in their respective conditioned media. After 24 hours incubation, 50 μl of 3-(4,5-dimethyl-2-thiazyl)-2,5-diphenyl-2H-tetrazolium bromide (MTT; 2 mg/ml) was added and further incubated for 4 hours. The supernatant was discarded and 100 μl of dimethylsulfoxide (DMSO) was added. Absorbance was read at 570 m with an ELISA Tecan SUNRISE™ plate reader. The control group consisted of cells without compounds and is referred to as 100% of viable cells. IC₅₀ was determined using PRISM® software.

Table 1 shows the effect (IC₅₀) of compounds on normal (PBML, Peripheral Blood Mononuclear Leukocytes; NHDF, Normal Human Dermal Fibroblast) and cancer (CAKI-2, human kidney cell; Hep-G2, human liver cell; PC-3, human prostate carcinoma cell; B16F10, murine melanoma cell and P815, murine mastocytoma cell) cell lines in a 24-hour cell culture. No cytotoxicity was observed in the presence of protein A. Some compounds, in particular dimeric triazine compounds, appeared to affect mostly cancer cells that may possess on their surfaces Fc receptors or immunoglobulin-like domains such as PC-3, DA-3, B16F10, and P815. The predictive utility of cell-based cytotoxicity assays to assess the potential in vivo anticancer activity of compounds with selected cancer cell lines is well established in the art and the use of whole cells, instead of isolated protein receptors or enzymes, provides a more reliable determination of activity. See, for example, Paull et al. J. Natl. Cancer Inst. 81:1088-1092 (1989); Monks et al. J. Natl. Cancer Inst. 83:757-766 (1991); Bandes et al. J. Natl. Cancer Inst. 86:770-775 (1994); and Kamate et al. Intl. J. Cancer 100:571-579 (2002).

TABLE 1
Effect of compounds on normal and cancer cell cytotoxicity in 24-hour cell culture.

| | IC₅₀ | | | | | | | |
|---|---|---|---|---|---|---|---|---|
| Compound | PBML | NHDF | CAKI-2 | Hep-G2 | PC-3 | B16F10 | DA-3 | P815 |
| 1 | 98 | >100 | >40 | 19 | 8.3 | 6 | 4 | 34 |
| 2 | >100 | >100 | >40 | >40 | 2.3 | 1 | 2 | 66.5 |
| 3 | >100 | >100 | >40 | >40 | >100 | 4 | — | 33 |
| 4 | 100 | >100 | — | — | 78 | 12 | — | 86 |
| 5 | >100 | >100 | — | — | 50 | 8.5 | 20 | >100 |
| 6 | >100 | >100 | — | — | 63 | 2.6 | 30 | >100 |
| 7 | >100 | >100 | — | — | 85 | 2.2 | — | 20 |
| 8 | >100 | >100 | — | — | 94 | 33 | 85 | >100 |
| 9 | >100 | >100 | — | — | >100 | >100 | — | >100 |
| 10 | >100 | >100 | — | — | 74 | 62 | >100 | >100 |
| 11 | — | — | — | — | 4.4 | 1.4 | — | 21.2 |
| 12 | — | — | — | — | 11 | 1.2 | — | 13.5 |
| 13 | — | — | — | — | 11 | 1.9 | — | 6.5 |
| 14 | — | — | — | — | 29.9 | 2.6 | — | 13.1 |
| 15 | — | — | — | — | 10.9 | 1.8 | — | 13.4 |
| 16 | — | — | — | — | 20.3 | 0.5 | — | 12.6 |
| 17 | — | — | — | — | 11.6 | 1.2 | — | 14.5 |
| 18 | — | — | — | — | 12.2 | 1.1 | — | 2.5 |
| 19 | — | 19.4 | — | — | 8.2 | 2.8 | 4.5 | 16.4 |
| Protein A | >20 | >100 | >40 | >40 | >100 | >100 | >100 | >100 |

Table 2 shows the effect (IC₅₀) of compounds on normal and cancer cell lines in a 72-hour cell culture. No cytotoxicity was observed in the presence of protein A.

TABLE 2
Effect of compounds on normal and cancer cell
cytotoxicity in 72-hour cell culture.

| | IC₅₀ | | | | | | | |
|---|---|---|---|---|---|---|---|---|
| Compound | PBML | NHDF | CAKI-2 | Hep-G2 | PC-3 | B16F10 | DA-3 | P815 |
| 1 | 45 | 16 | 15 | 23 | 8.3 | 6 | 4.2 | 34 |
| 2 | 10 | 16 | >40 | >40 | 4 | 1 | 1.9 | 2.2 |
| 3 | >100 | >100 | — | — | 15 | 6 | — | 3 |
| 4 | >100 | >100 | — | — | 37 | 37 | — | 32 |
| 5 | >100 | 78 | — | — | 26 | 39 | — | 44.7 |
| 6 | >100 | >100 | — | — | — | — | — | >100 |
| 7 | 79 | >100 | — | — | 8 | 3 | — | 1.3 |
| 8 | >100 | >100 | — | — | 100 | — | — | 92 |
| 9 | >100 | >100 | — | — | >100 | 91 | — | 43 |
| 10 | >100 | >100 | — | — | — | 36 | — | 36.6 |
| 11 | — | — | — | — | 4.9 | 6.3 | — | 4.8 |
| 12 | — | — | — | — | 3.3 | 2 | — | 2.8 |
| 13 | — | — | — | — | 6.1 | 2.2 | — | 2.1 |
| 14 | — | — | — | — | 15.6 | 10.2 | — | 7.8 |
| 15 | — | — | — | — | 4.3 | 2.9 | — | 3.3 |
| 16 | — | — | — | — | 5.1 | 3.5 | — | 2.4 |
| 17 | — | — | — | — | 4.0 | 3.6 | — | 1.5 |
| 18 | — | — | — | — | 10.3 | 0.6 | — | 0.7 |
| 19 | — | — | — | — | 5.7 | 6.2 | 13.4 | 8.6 |
| Protein A | >20 | >100 | — | — | — | — | — | — |

### Example 2

### Antitumor Effects of Compounds on a Primary B16F10 Melanoma Tumor

Female 6-8 week old C57BL/6 mice were injected intradermally on day 0 with 3.75×10⁴ B16F10 melanoma cells from ATCC (source of cell culture, Dr. I. J. Fidler). On day 14, tumors reached 80 mm and animals were randomized for treatments. Animals were then injected i.v. with saline (negative control) or compounds (50 mg/kg) on day 14, 16 and 18 or 10 mg/kg doxorubicin (Dox, positive control) on day 14. Mice were sacrificed on day 29. Body weight and tumor volume were recorded. Serial tumor volume was obtained by bi-dimensional diameter measurements with calipers, using the formula 0.4 (a×b²) where “a” was the major tumor diameter and “b” the minor perpendicular diameter.

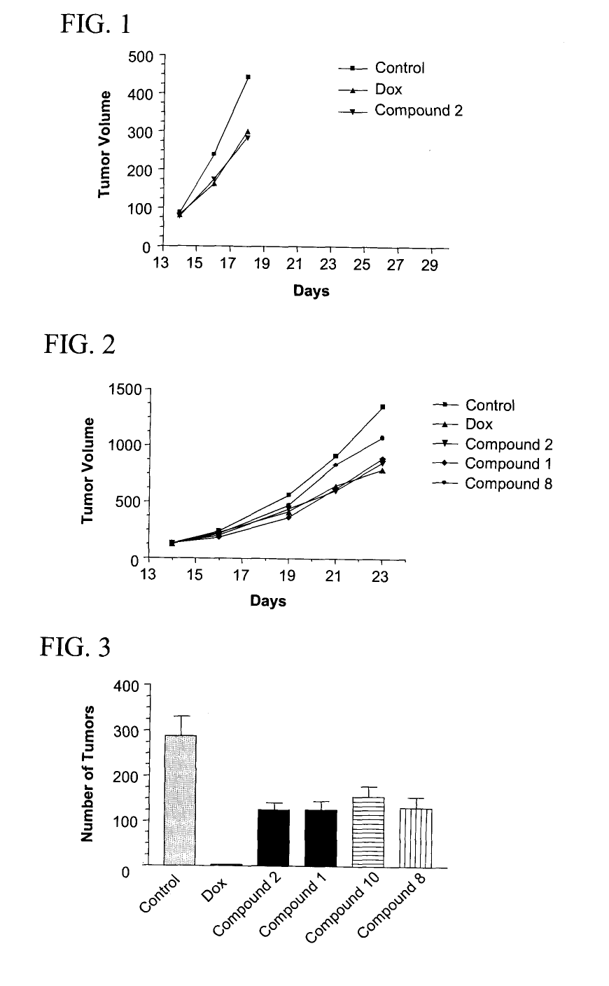

FIG. 1 shows the effect of compound 2 on primary tumor B16F10 cells. T/C is calculated as (Treated tumor volume/Control tumor volume)×100%. Compound 2 induced a weak reduction (T/C around 70%) of the tumor volume compared to the control. In this trial, however, this effect was comparable to doxorubicin.

FIG. 2 shows the effect of compound 1, 2 or 8 on primary tumor B16F10 cells. Compound 8 induced a weak reduction (T/C around 70%) of the tumor volume compared to the control. Compound 1 or 2 induced a significant reduction (T/C between 40% to 50%). Furthermore, the effect of compound 1 or 2 was comparable to doxorubicin.

### Example 3

### Antimetastatic Effects of Compounds on B16F10 Metastastic Tumors

Female 6-8-week old C57BL/6 mice were injected intravenously on day 0 with 1-2.5×10⁵ B16F10 melanoma cells from ATCC (source of cell culture, Dr. I. J. Fidler). B16F10 melanoma cells are a highly metastatic cell line which preferentially forms nodules in the lungs. Cells were cultured in DMEM supplemented with 10% fetal bovine serum. Animals were then injected i.v. with or without compounds (50 mg/kg) on day −3, −2, −1, 3, 5 and 7 and/or doxorubicin (10 mg/kg) on day 0. Fourteen days after inoculation, mice were sacrificed and their lungs were removed, rinsed in PBS, and placed in Bouin's fixative. The number of metastatic nodules (black colonies) on the surface of the lungs were counted.

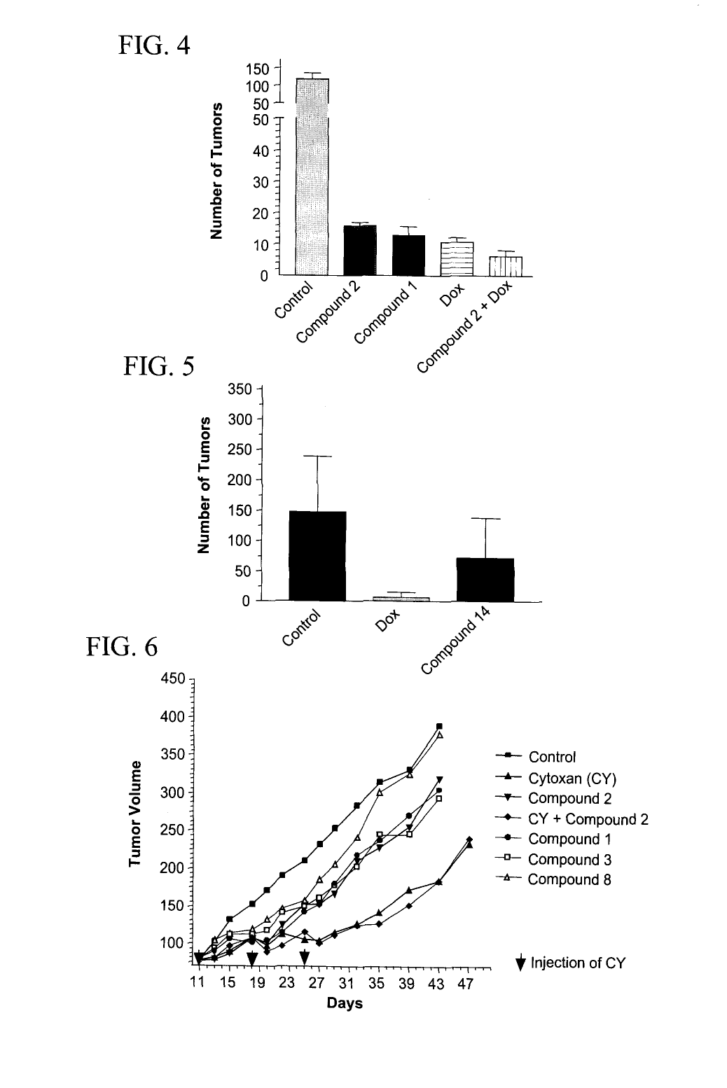

FIG. 3 shows the antimetastatic efficacy of compound 1, 2, 8 or 10. All compounds induced a significant inhibition (p<0.001) of the number of tumor nodules in lungs. Furthermore, in comparison to doxorubicin which induced significant toxicity as seen by a reduction (10%) of body weight, mice treated with the compounds displayed normal growth compared to control mice. Additionally, in a separate trial, FIG. 4 shows antimetastatic activity was undertaken with or without compounds in combination with a lower nontoxic concentration of doxorubicin (1 mg/kg) in a B16F10 melanoma model. Compound 2 induced a strong and significant reduction (87%) of the number of tumor nodules similar to doxorubicin (90%) when used alone. A stronger anticancer effect was observed when compound 2 is used in combination with doxorubicin (95%). Also, compound 14 induced a significant inhibition (50%; p<0.05) of the number of tumor nodules in lungs (FIG. 5).

### Example 4

### Antitumor Effects of Compounds on a Primary DA-3 Breast Tumor

The syngeneic tumor DMBA3 (DA-3, breast carcinoma model) arose from a preneoplastic lesion treated with 7,12-dimethylbenzanthracene in female BALB/c mice. DA-3 cells were grown as monolayer cultures in plastic flasks in RPMI-1640 containing 0.1 mM nonessential amino acids, 0.1 μM sodium pyruvate, 2 mM L-glutamine. This was further supplemented with 50 μM 2-mercaptoethanol and 10% fetal bovine serum. The DA-3 tumors were serially passage in vivo by intradermal inoculation of 5×10⁵ viable tumor cells to produce localized tumors in 6- to 8-week old BALB/c mice. The animals were then serially monitored by manual palpation for evidence of tumor. Mice were treated at day 11, 18 and 25 with cyclophosphamide (100 mg/kg) and at day 11, 12, 13, 15, 18, 20, 22, 25, 27 and 29 with compound 1, 2, 3 or 8 (50 mg/kg). Mice were sacrificed at day 43. Serial tumor volume was obtained by bi-dimensional diameter measurements with calipers, using the formula 0.4 (a×b²) where “a” was the major tumor diameter and “b” the minor perpendicular diameter. Tumors were palpable, in general, 7-10 days post-inoculation. The National Cancer Institute (USA) defines the product as effective if T/C is ≦40%.

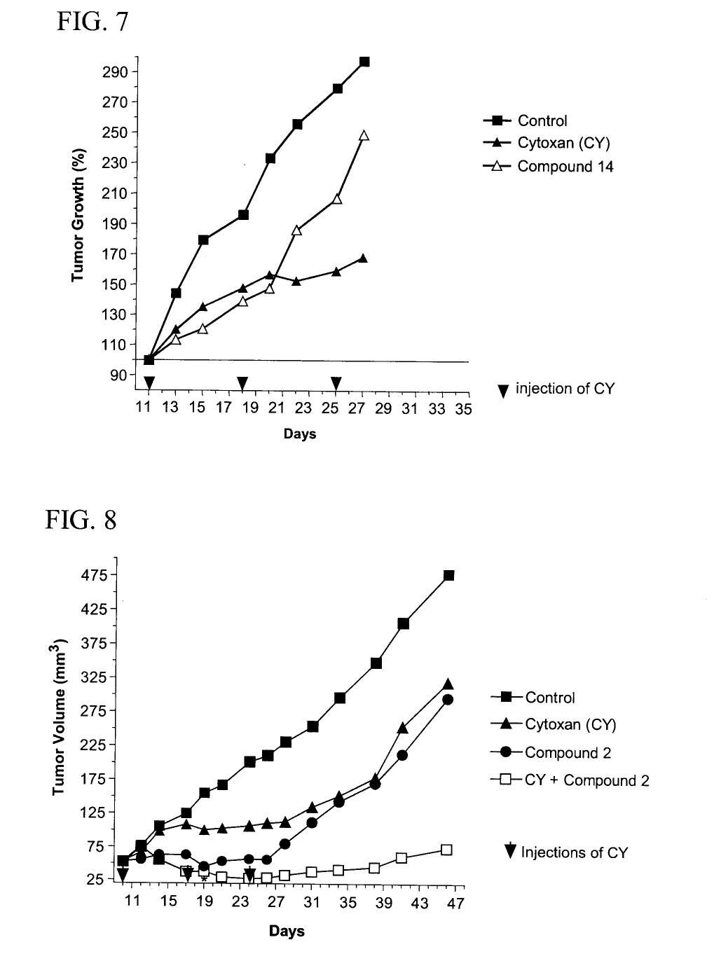

FIG. 6 shows the antitumor efficacy of compound 1, 2, 3 or 8 and the combination of cyclophosphamide and compound 2. All compounds except compound 8 induced a significant inhibition of the tumor volume. Compound 1 induced a significant (p<0.05) inhibition of tumor volume with a T/C between 28% to 74%. Compound 2 induced a significant (p<0.02) inhibition of tumor volume with a T/C between 22% to 79%. Compound 3 induced a significant (p<0.05) inhibition of tumor volume with a T/C between 37% to 64%. Furthermore, in comparison to cyclophosphamide which induces significant (p<0.03) inhibition of tumor volume with a T/C between 18% to 43%, mice treated with the combination of cyclophosphamide and compound 2 also induced a significant (p<0.02) inhibition of tumor volume with a T/C between 16% to 47%. FIG. 7 shows the antitumor efficacy of compound 14 which induced 25% to 60% inhibition of tumor volume.

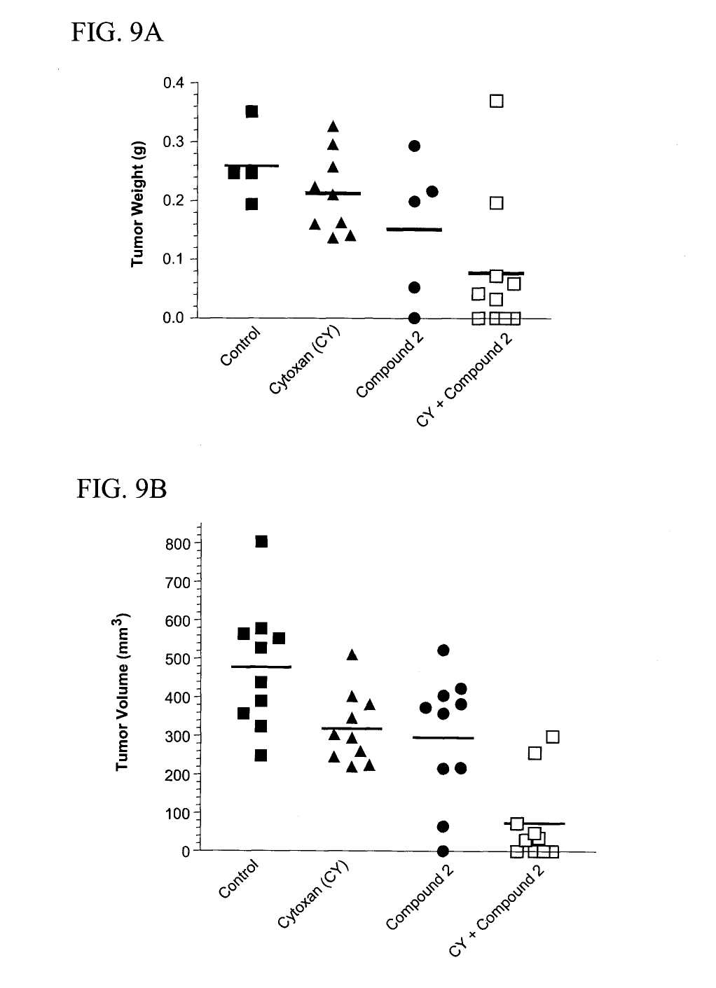

In other trials, mice were treated with one intratumoral injection of compound 2 or cyclophosphamide (three doses) or intratumoral injection of compound 2 in combination with cyclophosphamide. FIG. 8 shows the antitumor efficacy of intratumoral injection of compound 2 with or without cyclophosphamide combined. Intratumoral injection of compound 2 induced a significant (p<0.05) inhibition of tumor volume with a T/C between 25% to 70% accompanied with one total tumor regression at day 46. Cyclophosphamide induced a weak inhibition of tumor volume with a T/C between 55% to 80%. Mice treated with the combination of cyclophosphamide and intratumoral injection of compound 2 demonstrated a significant (p<0.001) inhibition of tumor volume with a T/C of 10% accompanied with four total tumor regressions at day 46. FIG. 9 shows the tumor's weight (FIG. 9A) and volume (FIG. 9B) at the end of the trial (day 46).

### Example 5

### Antitumor Effects of Compounds on a Primary P815 Mastocytoma Tumor

The syngeneic tumor P815 is a DBA/2 (H-2^{d})-derived mastocytoma obtained from ATCC (TIB64). P815 cells were grown in DMEM containing 10% fetal bovine serum. At day 0, 5×10⁵ viable P815 cells were intradermally injected to produce localized tumors in 6- to 8-week old DBA/2 mice. The animals were then serially monitored by manual palpation for evidence of tumor. Mice were then treated every day with intraperitoneal injection of vehicle (negative control), acetylsalicylic acid (positive control, 50 mg/kg), or compound 2 (50 mg/kg). Mice were sacrificed at day 23. Serial tumor volume was obtained by bi-dimensional diameter measurements with calipers, using the formula 0.4 (a×b²) where “a” was the major tumor diameter and “b” the minor perpendicular diameter. Tumors were palpable, in general, 3-5 days post-inoculation.

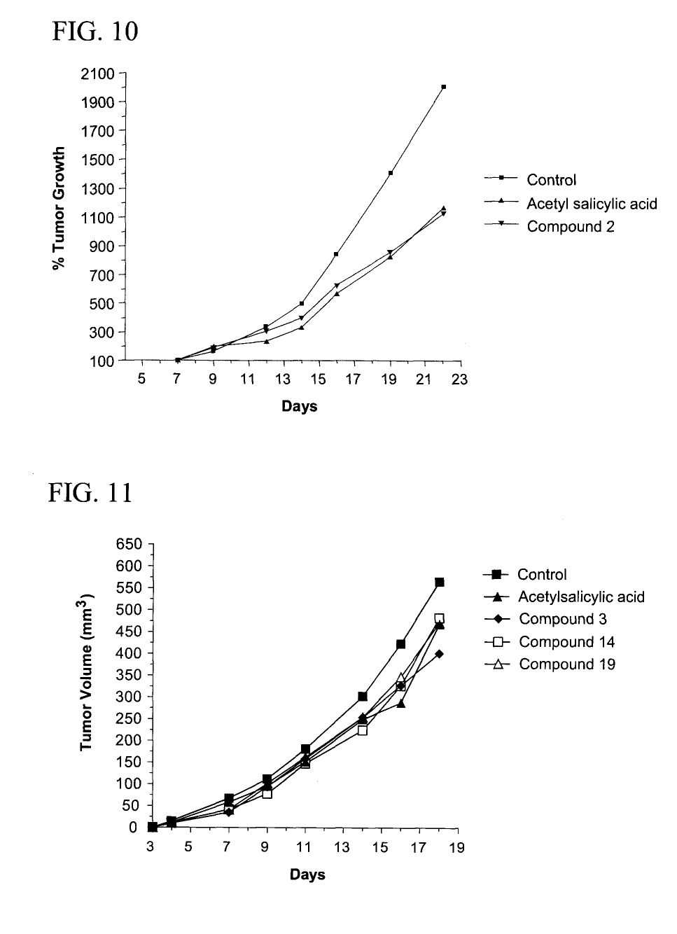

FIG. 10 shows the effect of compound 2 on primary tumor P815 cells. Compound 2 induced a significant reduction (T/C between 40% to 50%) of tumor growth. Furthermore, the effect of compound 2 was comparable to soluble acetylsalicylic acid.

FIG. 11 shows the effects of intraperitoneal injection of vehicle (negative control), acetylsalicylic acid (positive control, 50 mg/kg), compound 3 (50 mg/kg), compound 14 (25 mg/kg), and compound 19 (50 mg/kg) on primary tumor P815 cells. All compounds induced a reduction (T/C between 60% to 80%) of tumor growth comparable to soluble acetylsalicylic acid.

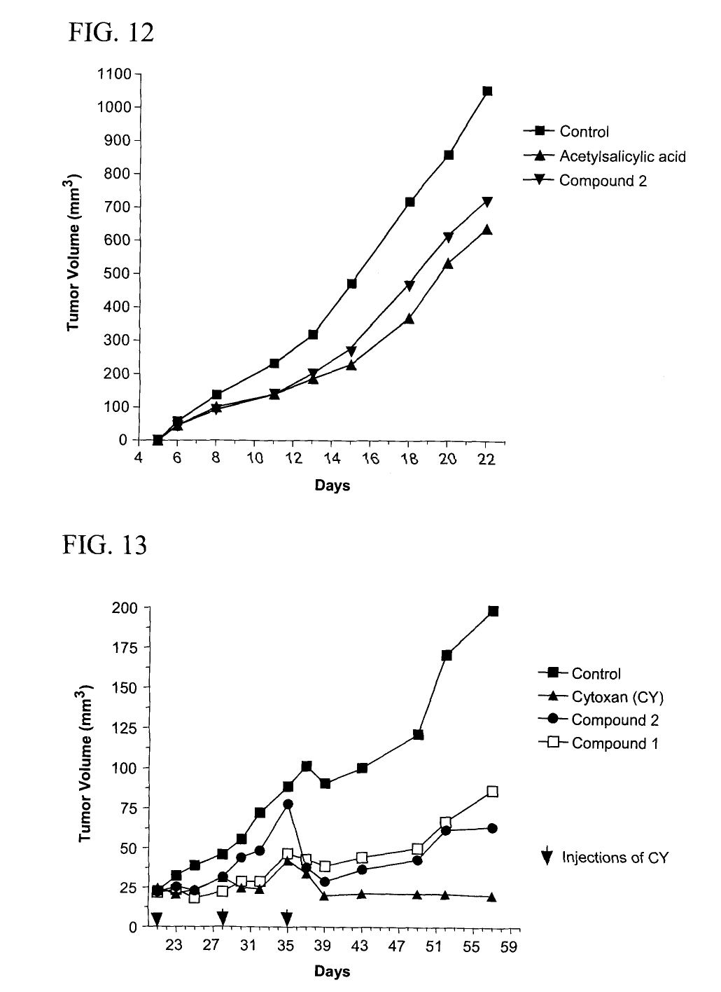

In another trial, mice were treated with daily oral administration of acetylsalicylic acid or compound 2 at 50 mg/kg. FIG. 12 shows the effects of oral administration of compounds on primary tumor P815 cells. All compounds induced a reduction (T/C between 30% to 60%) of tumor growth. Furthermore, compound 2 was comparable to soluble acetylsalicylic acid.

### Example 6

### Antitumor Effects of Compounds on Xenograft Human Prostate PC-3 Tumor

The xenogenic human prostate tumor PC-3 was obtained from ATCC (CRL1435). PC-3 cells were grown in RPMI-1640 containing 10% fetal bovine serum. At day 0, 50 μl of viable PC-3 (1.5 to 2×10⁶) cells were intradermally injected to produce localized tumors in 6- to 8-week old male CD1 nu/nu mice. The animals were then serially monitored by manual palpation for evidence of tumor. When the tumors reached a satisfactory volume, mice were randomized and then treated four, three, and three times per week in the first, second, and third week, respectively, with intravenous injection of vehicle (negative control), cyclophosphamide (positive control, 100 mg/kg), compound 1 (50 mg/kg), compound 2 (50 mg/kg), or compound 8 (50 mg/kg). Mice were sacrificed between days 56 to 65. Serial tumor volume was obtained by bi-dimensional diameter measurements with calipers, using the formula 0.4 (a×b²) where “a” was the major tumor diameter and “b” the minor perpendicular diameter.

FIG. 13 shows the effects of compound 1, compound 2 and cyclophosphamide on xenograft human prostate PC-3 tumor cells. Compound 1 induced a significant reduction (T/C between 1% to 52%) of tumor growth. Compound 2 induced a significant reduction (T/C between 16% to 84%) of tumor growth. Cyclophosphamide induced a significant reduction (T/C between 1% to 23%) of tumor growth.

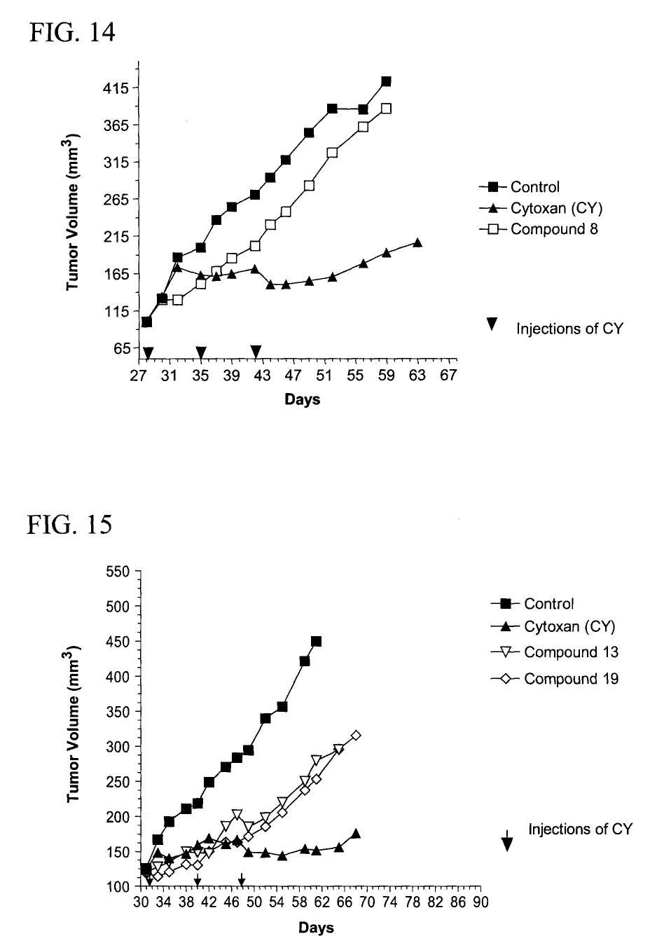

FIG. 14 shows the effects of compound 8 and cyclophosphamide on xenograft human prostate PC-3 tumor cells. Compound 8 induced a significant reduction (T/C between 29% to 75%) of tumor growth. Cyclophosphamide induced a significant reduction (TIC between 1% to 52%) of tumor growth.

FIG. 15 shows the effects of compound 13, compound 19, and cyclophosphamide on xenograft human prostate PC-3 tumor cells. Compound 13 induced a significant reduction (T/C between 8% to 36%) of tumor growth. Compound 19 induced a significant reduction (T/C between 20% to 68%) of tumor growth. Cyclophosphamide induced a significant reduction (T/C between 1% to 50%) of tumor growth.

Patents, patent applications, and other publications cited herein are incorporated by reference in their entirety.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. Any of the three transitions can be used to claim the invention.

It should be understood that an element described in this specification should not be construed as a limitation of the claimed invention unless it is explicitly recited in the claims. Thus, the claims are the basis for determining the scope of legal protection granted instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of the individual elements disclosed herein are considered to be aspects of the invention; similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method of treating a mammal with cancer, comprising administration to said mammal of a therapeutically effective amount of a dimeric triazine compound described by Formula I: 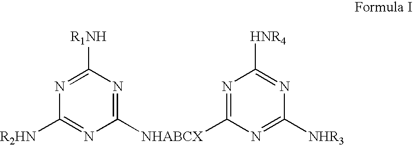
where A=—(CH₂)ₙ—, n=0, 1, 2, 3 or 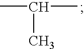 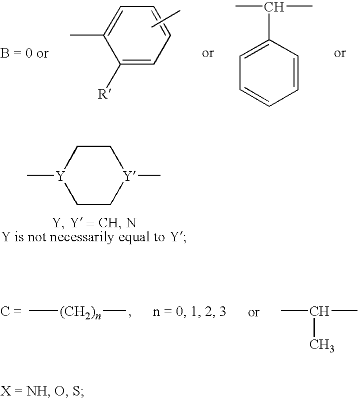
R′=hydrogen or C₁₋₄ alkyl, C₁₋₄ N-methylaminoalkyl or N,N-dimethylaminoalkyl;
A is not necessarily equal to C;
and wherein R₁, R₂, R₃ and R₄ are independently selected from the group consisting of hydrogen, C₂₋₆ alkyl or alkenyl, C₂₋₆ hydroxyalkyl, C₂₋₆ aminoalkyl, trifluoromethyl, pentafluoroethyl, phenyl, naphthyl, benzyl, biphenyl, phenethyl, piperazinyl, N-methylpiperazinyl, N-ethylpiperazinyl, morpholinyl, piperidinyl, methylpiperidinyl, ethylpiperidinyl, indenyl, 2,3-dihydroindenyl, C₄-C₇ cycloalkyl or cycloalkenyl, indoyl, methylindoyl, ethylindoyl, and substituted five-membered aromatic heterocyclic rings of the following formulas: 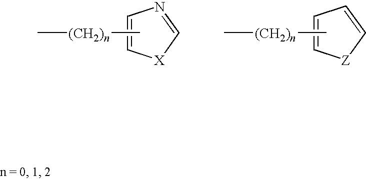
X is defined as above and Z=NH, CH₂
or substituted phenyl rings of the following formulas: 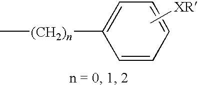
X and R′ are defined as above 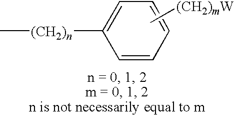
W=hydrogen, CH₃, NH₂, COOR′, OR′ 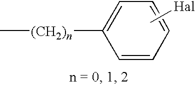
Hal=Halogen (F, Cl, etc.) 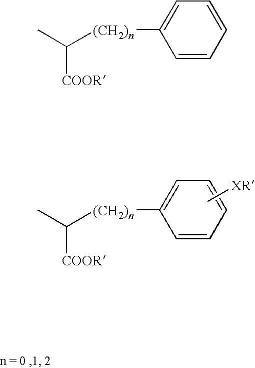
X and R′ are defined as above.

2. A method of treating a mammal with cancer, comprising administration to said mammal of a therapeutically effective amount of a monomeric triazine compound described by Formula II: 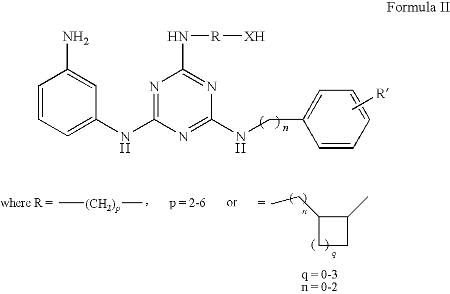
X═NH, O, or S
and R′=NH₂, OCH₃, F or Cl
or where the group —R—XH is replaced by 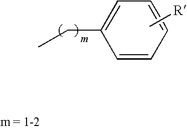
in such a case, the general formula becomes: 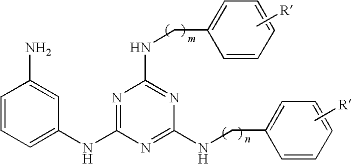
wherein R′ is defined as above but, if two R′ substituents are present in the same compound, both R′ substituents may be the same (amino, methoxy, fluorine) or one R′ substituent can be an amino group or fluorine atom while the second is a methoxy group and m is not necessarily equal to n;
or in the case where R′ is meta amino and n=0 then —R—XH may be replaced such that the general formula becomes: 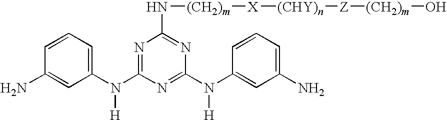
wherein m=1-2, n=2-4, X═CHY, O, S; Y═H, OH; and Z=zero, O, S
or in the case where —R—XH is replaced by a hydrogen atom such that the general formula becomes: 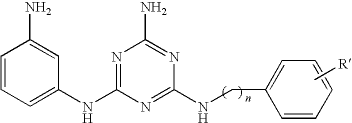
wherein R′ and n are defined as above.

3. A method of treating a mammal with cancer, comprising administration to said mammal of a therapeutically effective amount of a compound selected from the group consisting of:

| Compound No. | Structure |
|---|---|
| 1 | 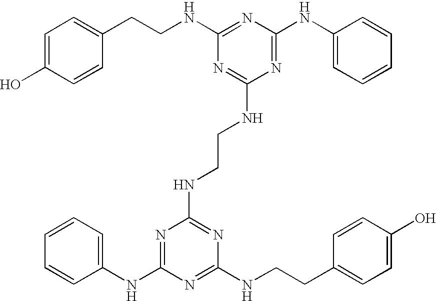 |
| 2 | 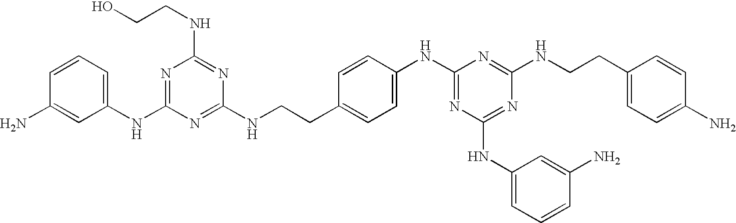 |
| 3 | 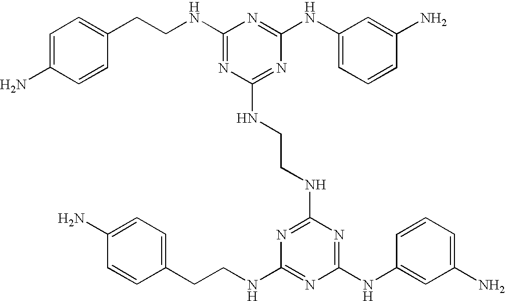 |
| 4 | 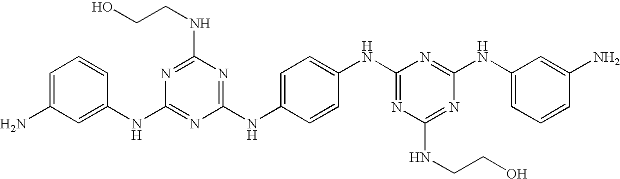 |
| 5 | 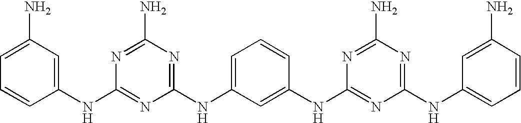 |
| 6 | 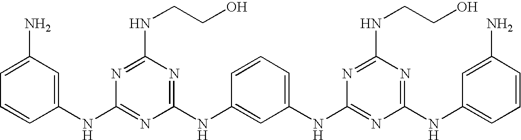 |
| 7 | 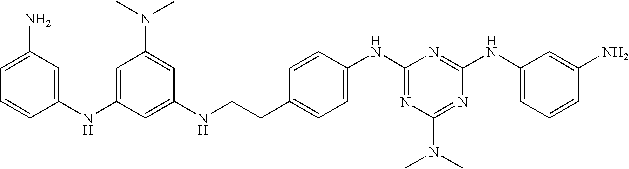 |
| 8 | 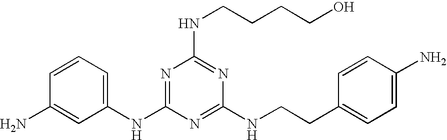 |
| 9 | 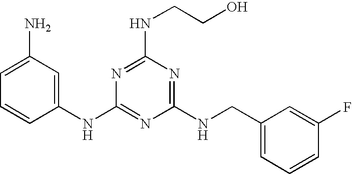 |
| 10 | 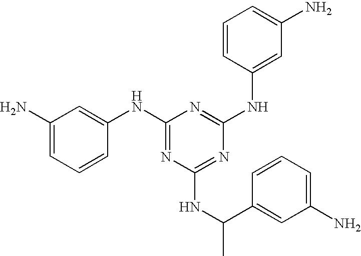 |

4. A composition comprised of at least one compound according claim 1, wherein said compound is combined with a pharmaceutically acceptable carrier.

5. The composition according to claim 4, wherein said carrier solubilizes said compound and is selected from the group consisting of alcohols, polyol solvent, and aqueous solutions of a mono- or disaccharide.

6. The composition according to claim 4, wherein said carrier is dimethylacetamide.

7. The composition according to claim 4 further comprised of a chemotherapeutic agent.

8. The composition according to claim 7, wherein said chemotherapeutic agent is selected from the group consisting of decarbazine, doxorubicin, daunorubicin, cyclophosphamide, vinblastine, vincristine, bleomycin, etoposide, topotecan, irinotecan, taxotere, taxol, 5-fluorouracil, methotrexate, gemcitabine, cisplatin, carboplatin, and chlorambucil.

9. The composition according to claim 4 further comprised of a cytokine.

10. A method of treating a patient with cancer, said method comprising administration to said patient of a therapeutically effective amount of a compound according to claim 1.

11. The method of treating a patient with cancer according to claim 10, wherein said cancer is characterized by cancer cells which express cell surface Fc receptors.

12. The method of treating a patient with cancer according to claim 10, wherein said cancer is metastatic melanoma.

13. (canceled)
